(19) United States
(12) Patent Application Publication
(10) Pub. No.: US 2015/0122696 A1
(43) Pub. Date: May 7, 2015

(54) PacketPouch

(71) Applicants: Rachel Ellen Sankey, Martinez, GA (US); Xavier Eugene Wade, Augusta, GA (US)

(72) Inventors: Rachel Ellen Sankey, Martinez, GA (US); Xavier Eugene Wade, Augusta, GA (US)

(21) Appl. No.: 14/070,539

(22) Filed: Nov. 3, 2013

(51) Int. Cl. A61F 17/00

(52) U.S. Cl. CPC A61F 17/00
USPC 206/570

(57) ABSTRACT

The invention provides an improvement to current medical packaging. The back of the package has adhesive that when the protective covering is removed the package can be secured to any surface. This adhesive turns any surface into storage allowing proper placement of medical equipment for further use on a single patient. The package may if desired also have an additional pouch inside. This pouch allows for a second option for storage. Medical equipment and supplies are easily accessible when needed, properly stored without falling on the floor or cross-contaminating multiple surfaces. This design improvement assures that all equipment or supplies are also accounted for during procedures. It assists in eliminating waste and lost supplies.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

The addition of a type of adhesive to the existing packaging of medical or other devices is an improvement on existing packaging. This adhesive when applied to the package will allow the package to be adhered to any surface. A double-sided adhesive or the like will need to be placed on the package on the backside at or near the top of the package. When the equipment is needed, the package will be opened so as to allow for the equipment to be placed back into open package. The adhesive covering will then be removed and the package adhered to a surface allowing the equipment to be stored easily and accessed easily if reuse is desired. When equipment is no longer needed, the package can be removed from surface and discarded appropriately. If package is unable to be opened so as to be reused for storage and optional pouch may be added to the existing packaging during manufacture to be used as storage on any surface.

What is claimed is:

1. An article of manufacture for storage of medical instruments or equipment improvement comprising:
Existing packaging for medical equipment;
Adhesive;
Cover for adhesive to be removed when attaching to surface; and
Optional pouch for additional storage.
